(19) United States
(12) Patent Application Publication
(10) Pub. No.: US 2003/0157162 A1
(43) Pub. Date: Aug. 21, 2003

(54) Liposome-encapsulated opioid analgesics

(76) Inventors: Lisa A. Krugner-Higby, Belleville, WI (US); Timothy D. Heath, Madison, WI (US); Lesley J. Smith, Madison, WI (US)

Correspondence Address: Licata & Tyrrell P.C., 66 E. Main Street, Marlton, NJ, 08053, US

(21) Appl. No.: 10/350,207

(22) Filed: Jan. 22, 2003

Related U.S. Application Data
(60) Provisional application No. 60/350,640, filed on Jan. 22, 2002.

(51) Int. Cl. A61K 9/127; A61K 31/485

(52) U.S. Cl. 424/450; 514/282

(57) ABSTRACT

Liposome-encapsulated opioid formulations and methods of use for long-term analgesic activity in animals are provided.

### INTRODUCTION

This application claims benefit under 35 U.S.C. §119 of U.S. provisional application Serial No. 60/350,640, filed on Jan. 22, 2002 whose contents are incorporated herein by reference in its entirety.

### BACKGROUND OF THE INVENTION

Adequate pain relief in both laboratory animals and in humans following surgical procedures is constantly sought. Many of the opiate drugs commonly used for pain relief, such as morphine sulfate, must be administered every two to four hours or sometimes even more frequently. In the case of laboratory animals used in medical research or in the case of veterinary medicine, there are barriers to use of the shorter-acting opiates such as morphine sulfate that require such frequent dosing, barriers that include the access to medications during night and evening hours when staff are not available at these facilities, as well as the possible drug diversion by personnel. In the case of humans, longer-acting opiate drugs have been put to use as they are associated with fewer side effects and lower potential for abuse. Therefore, longer-acting opiate drugs have been sought that could be used in animals as well as in humans.

Liposome encapsulation is a technique that has been used in human medicine to develop longer-acting analgesic drug formulations. Liposome-encapsulated morphine sulfate has been developed. Mice injected subcutaneously with liposome-encapsulated morphine sulfate maintained continuous plasma concentrations of greater than or equal to 1 μg/ml for six days after a single injection (Kim, et al. (1993) Cancer Chemother. Pharmacol. 33:187-190). A single dose of 250 μg of liposome-encapsulated morphine sulfate administered epidurally to rats had significant analgesic effects for 3 to 4 days as measured by hot plate testing (Kim, et al. (1996) Anesthesiology 85:331-338). Dogs administered liposome-encapsulated morphine sulfate had effective analgesia as measured by skin-twitch response latency for up to 60 hours after a single epidural dose of 30 mg/3 ml (Yaksh, et al. (1999) Anesthesiology 90: 402-412). Improved formulations for providing long-term analgesic activity are needed.

### SUMMARY OF THE INVENTION

An object of the present invention is a liposome-encapsulated opioid formulation for long-term analgesic activity comprising opioid encapsulated in a liposome by rehydration/dehydration method. This rehydration/dehydration method involves suspending opioid in a buffer to form a opioid-buffer mixture; overlaying the opioid-buffer mixture onto a film of lipid; sonicating the opioid-buffer mixture and the lipid to form a liposome mixture; freezing the liposome mixture by mixing the liposome mixture over a slurry of dry ice and isopropanol; and freeze-drying the mixture for storage in a freezer until the liposomes are rehydrated in sterile water.

Another object of the present invention is a method for producing liposome-encapsulated opioid for long-term analgesic activity which comprises suspending opioid in a buffer to form a opioid-buffer mixture; overlaying the opioid-buffer mixture onto a film of lipid; sonicating the opioid-buffer mixture and the lipid to form liposomes; freezing the liposome mixture by mixing the liposome mixture over a slurry of dry ice and isopropanol; and freeze-drying the mixture for storage in a freezer until the liposome mixture is rehydrated in sterile water.

Another object of the present invention is a method for producing liposome-encapsulated opioid for long-term analgesic activity which comprises suspending opioid in a buffer to form a opioid-buffer mixture; overlaying the opioid-buffer mixture onto a film of lipid; freezing opioid-buffer mixture and film of lipid; and thawing to form liposomes.

Another object of the present invention is a method of producing long-term analgesic activity in an animal which comprises administering to an animal an effective dose of the liposome-encapsulated oxymorphone formulation of the present invention so that analgesia is produced for a longer period of time than analgesia that results from administration of an effective dose of a non-liposome-encapsulated oxymorphone formulation.

Yet another object of the present invention is a method for reducing the dose-limiting toxicity of oxymorphone hydrochloride in an animal comprising administering to an animal an effective dose of the liposome-encapsulated oxymorphone formulation of the present invention.

### DETAILED DESCRIPTION OF THE INVENTION

It has now been found that opioid drugs such as oxymorphone, morphine, butorphanol, and hydromorphone can be encapsulated with high efficiency into liposomes. These liposomes, when administered to animals, provided long-term analgesia. These compositions have use in both animals and humans as a long-term analgesic formulation of opioids.

Oxymorphone is an opioid agonist that is ten times more potent than morphine and, unlike another opioid agonist-antagonist, buprenorphine, has no ceiling effect to the analgesia it provides. Further, oxymorphone is associated with less histamine release than morphine (Robinson, et al. (1988) Am. J. Vet. Res. 49:1699-1701; Smith, et al. (2001) J. Am. Vet. Med. Assoc. 218:1101-1105). Therefore, oxymorphone is a desirable drug for use in providing analgesia in humans as well as animals. Oxymorphone is currently approved for use in the Unites States as an analgesic, but, like morphine, the duration of action of the drug is only a few hours when it is administered as currently formulated as an injectable solution or a suppository. Formulations of the opiates that will extend their activity in the body have been sought and are provided by the present invention.

Liposomes are a formulation that is now well-known in the art. However, there are many different ways to produce liposomes and the method of production can affect the efficiency with which a drug is encapsulated and the activity of the drug once it is encapsulated. Liposome formulations for any given drug must be carefully chosen and optimized for that drug. It is not possible to predict which method will be useful for which drug and whether once encapsulated the release characteristics of the drug from the liposome will be compatible with the pharmacological properties of the drug. The activity of the drug once encapsulated can be modified such that the drug is no longer as effective, i.e., it has an altered efficacy profile. Therefore, even drugs with well-established pharmacological profiles and dose-response relationships must be tested in vivo once encapsulated to ensure that the drug is still capable of producing the desired pharmacological activity and to elucidate the dose-response profile of the encapsulated drug.

Studies were performed wherein oxymorphone liposomes were prepared using a simple vortex mixer/shaker procedure. In this method, 1 ml of oxymorphone hydrochloride was added to a 2 dram vial containing a standard lipid mixture (2.8 mM 1,2-dipalmitoyl-sn-glycero-3-[phospho-rac-(1-glycerol)] (sodium salt), 13.2 mM 1,2-dioleoyl-sn-glycero-3-phosphocholine, and 19.9 mM cholesterol), 2.4 mM triolein and tricaprylin (4:1 ratio), and 0.78 ml/L sterile water suspended in 1 ml chloroform. The vial was shaken at high speed with a vortex mixer for 10 minutes. The mixture was rapidly pipetted into two vials containing 2.5 ml continuous aqueous buffer (glucose, 32 mg/ml; free-base lysine, 40 mM). The vials were shaken with a vortex mixer for 20 seconds. The contents of the vials were then added to 45 ml of the continuous aqueous phase in a 250 ml flask and placed under a constant flow of nitrogen gas (7 L/minute) for 20 minutes to evaporate the chloroform. The lipid-encapsulated oxymorphone particles were then isolated by centrifugation at 600× g for 5 minutes, washed three times in normal saline, and stored at 2 to 8° C. Control liposomes were produced as well that contained 6% wt/vol sucrose.

Using this procedure, the encapsulation efficiency of the oxymorphone was low, only 2.5%. Therefore, liposomes containing oxymorphone were thus prepared using other methodology. In one embodiment of the invention, oxymorphone hydrochloride was encapsulated using a dehydration/rehydration method. Oxymorphone hydrochloride was suspended in citrate buffer and overlaid on a thin film of egg phosphatidyl choline (PC) in an 18 mm test tube. The test tube was sonicated to suspend the egg PC in the oxymorphone solution. The mixture was transferred to a sterile round-bottomed flask and frozen using a slurry of dry ice and isopropanol. Then the contents of the flask were freeze-dried overnight and stored at −20° C. until rehydration. The liposomes were prepared by rehydrating the lyophilized preparation for 0.5 hour with 0.5 ml of distilled sterile water for irrigation, and diluted with 9.5 ml of sterile physiologic saline with 10 mM acetate buffer pH 4.0 (saline-acetate buffer). The liposomes were transferred to a 15 ml conical centrifuge tube and sedimented at 1,000 g for 10 minutes. The excess buffer was removed and the resulting pellet, which contained about 0.5 ml of packed liposomes, was resuspended and sedimented twice more, and was finally resuspended and stored at 5° C. in 2 ml of saline-acetate buffer (pH 4.0). Oxymorphone in the liposome preparations was quantitated by suspending 200 μl of the appropriately diluted liposome preparation in a solvent solution containing 600 μl of methanol and 200 μl of chloroform, and agitating the solution gently on a test tube vortex. The solution was placed in a cuvette and the concentration of oxymorphone was determined for the absorbance using a molar extinction coefficient of 1.437 cm⁻¹ at an absorbance of 281 nm. Saline-acetate buffer suspended in the same solvent solution was used as a blank. Encapsulation efficiency for oxymorphone-containing liposomes was 69% and 32% for the first and second batches of liposomes, respectively.

The encapsulation efficiency for morphine sulfate was not as high as that for oxymorphone. The average efficiency for morphine encapsulation in the 3 batches used in these studies was 16%. Thus, oxymorphone encapsulation was far superior to the efficiency of morphine encapsulation.

Other opioid drugs such as hydromorphone and butorphanol have also been successfully encapsulated using the above dehydration/rehydration method. For example, hydromorphone was encapsulated with an efficiency of almost 50% (46.8%).

In another embodiment of the invention, the oxymorphone hydrochloride was encapsulated using a modified version of the dehydration/rehydration method. Oxymorphone hydrochloride was suspended in citrate buffer and overlaid on a thin film of egg phosphatidyl choline (PC) in an 18 mm test tube. The mixture was transferred to a sterile round-bottomed flask and frozen using a slurry of dry ice and isopropanol. The mixture was thawed and the liposomes were transferred to a 15 ml conical centrifuge tube and sedimented at 1,000 g for 10 minutes. Subsequent resuspension and sedimentation steps were similar to the dehydration/rehydration method.

In vitro release kinetics of the liposome-encapsulated oxymorphone preparation were determined via diffusion of the released drug from dialysis sacs. The preparation of liposome-encapsulated oxymorphone leaked and diffused from the dialysis sacs and into the buffer solution over a period of 5 days. After 48 hrs, 67% of the oxymorphone hydrochloride had been released into the buffer, and 96% had been released by 5 days.

Pharmacokinetics were determined in rats by taking blood samples (1 ml) prior to injection of the liposomes, 4 hours after injection, and then daily for 7 days post-injection. The performance of the liposome-encapsulated opioids was tested in vivo in parrots, in neuropathic and visceral pain models in rats and pharmacokinetics were determined in a dog model. In the neuropathic pain model, liposome-encapsulated oxymorphone was injected subcutaneously into rats (300 g) immediately prior to surgical ligation of the sciatic nerve, an animal model for neuropathic pain that peaks within 7 days. Foot withdrawal times in response to a point heat source were determined in the rats before surgery and then once a day for 7 days after surgery. Control animals were injected with liposome-encapsulated sucrose.

Control rats (non-ligated) administered either liposome-encapsulated sucrose (Group 4) or liposome-encapsulated morphine (Group 5) exhibited no change in thermal withdrawal latency from baseline to day 7. These results indicate that liposome-encapsulated morphine, at least at the doses administered herein, did not affect normal thermal threshold. Interestingly, the control rats administered liposome-encapsulated oxymorphone (Group 6) exhibited a small but statistically significant (p=0.04) increase in thermal withdrawal latency from day 4 through 7. This analgesic or sedative effect in non-hyperalgesic rats may have been due to the relatively high dose, compared with morphine on a receptor affinity basis, chosen for the liposome-encapsulated preparation of oxymorphone.

Ligated rats administered liposome-encapsulated sucrose (Group 1) exhibited thermal hyperalgesia, an indicator of the development of neuropathic pain, by day 4 following sciatic nerve ligation, with maximal hyperalgesia by postoperative day 7. At both days 4 and 7, thermal withdrawal latencies for Group 1 were significantly lower than in any other group (p<0.001) and significantly lower than baseline values within Group 1 (p<0.001). These results indicate that thermal hyperalgesia was established via sciatic nerve ligation and that the liposomal vehicle used did not impart any analgesic effects.

Rats administered liposome-encapsulated oxymorphone (Group 2) at the time of sciatic nerve ligation showed no significant change in thermal withdrawal latencies at day 4 compared with baseline (p=0.85). Thermal withdrawal latencies were significantly longer at day 7 in Group 2 (p=0.04), compared with postoperative day 4, indicating that the rats showed no evidence of thermal hyperalgesia. The increase in foot withdrawal latency observed in liposome-encapsulated oxymorphone rats at day 7 may be due to progressively increasing release of the opioid by day 7. This result was also seen in non-ligated control rats. Sedative effects of the oxymorphone appeared to not play a significant role as subjective assessment of sedative effects did not indicate sedation had occurred in the rats. Rats given liposome-encapsulated morphine (Group 3) at the time of surgery showed no significant change in thermal withdrawal latencies throughout the course of the experiment (p=0.68). These results indicate that hyperalgesia from sciatic nerve ligation was prevented for up to 7 days by one treatment of liposome-encapsulated morphine or oxymorphone. These data indicate that liposome-encapsulated oxymorphone administered at the time of surgery provided pre-emptive analgesia by preventing plastic changes within the nervous system that lead to central sensitization (Woolf and Chong (1993) Anesth. Analg. 77:362-379). Therefore, although the liposome-encapsulated morphine was also shown to have long-term analgesic effects, the effects seen with oxymorphone were superior in terms of their analgesic properties. In addition, control rats treated with liposome-encapsulated oxymorphone had an increase in thermal threshold, indicating that this treatment not only prevented hyperalgesia, but may also be analgesic in non-neuropathic rats.

Serum concentrations of oxymorphone were detectable up to three days after a single injection of the liposome-encapsulated preparation. Serum concentrations after injection of the liposome-encapsulated oxymorphone at day one (1.23±0.4 ng/ml) were similar to serum concentrations of oxymorphone measured at 4 hours (1.52 34±0.7 ng/ml) when rats were given a comparable dose of standard oxymorphone s.c. General dosing recommendations for standard oxymorphone are that it be given to rats every 4 hours to maintain analgesic serum concentrations (Hawk and Leary (1999) In: Formulary for Laboratory Animals. 2^{nd} Ed. Iowa State University Press, Ames, Iowa pg. 19). Because serum concentrations were fairly equivalent at one day after injection of liposome-encapsulated oxymorphone compared to serum concentrations at 4 hours after injection with standard oxymorphone, these data indicate that therapeutic concentrations of drug were maintained for at least 24 hours after injection of the liposome-encapsulated formulation. Urine concentrations of oxymorphone were highest 24 hours after injection, but were detectable through day 7 and had only decreased to approximately 50% of the 24-hour level by day 7. In urine collected via metabolism cages in non-treated rats, oxymorphone detection was 0.263±0.025 ng/ml; thus background interference in urine for oxymorphone detection was insignificant. Therefore, these data demonstrated that liposome-encapsulated oxymorphone was an effective and safe analgesic treatment in a well-established animal model for chronic human pain.

Experiments using liposome-encapsulated oxymorphone indicate that one injection of the preparation was as good or better than repeated injections of standard oxymorphone for treating pain associated with intestinal surgery in rats (i.e., visceral pain model). The intestinal surgery model was chosen to evaluate the efficacy of liposome-encapsulated oxymorphone for several reasons. The surgical techniques used were similar to those used in a broad range of both experimental and clinical surgeries. It was already known that the surgeries were associated with considerable discomfort in rats and that the standard pharmaceutical preparation of oxymorphone was effective at controlling pain in these rats when administered on a repeated basis. There was no difference in pain scores between rats that had intestinal resection as the surgical procedure compared with those that had intestinal transection procedures (p=0.12). Therefore, data for resection and transection rats are combined in all subsequent analyses. Data using pain scores, based on behavioral observations, indicated that a single injection of 1.2 mg/kg liposome-encapsulated oxymorphone (Group L1) given prior to surgery was at least as effective as doses of 0.3 mg/kg of standard oxymorphone hydrochloride given every 4 hours (Group S1) at reducing or relieving post operative pain in rats (p=0.18), and that a single injection of 1.6 mg/kg liposome-encapsulated oxymorphone (Group L2) was at least as effective as doses of 0.3 mg/kg of standard oxymorphone given every 8 hours (Group S2) (p>0.05) . The lower dose of liposome-encapsulated oxymorphone (1.2 mg/kg) (Group L1) was actually associated with lower. pain index scores than the higher dose (1.6 mg/kg) (Group L2) (p=0.027) . Also, rats that were given 0.3 mg/kg of standard oxymorphone every 4 hours (Group S1) had lower pain index scores than rats that were given 0.3 mg/kg standard oxymorphone every 8 (Group S2) hours (p<0.01). Pain index scores for rats given liposome-encapsulated oxymorphone were equal, and in some cases lower than, rats administered the standard pharmaceutical preparation of oxymorphone. Side effects such as sedation or agitation were not observed in rats given liposome-encapsulated oxymorphone.

In general, rats that have had resections and are fed parenterally lose body weight for 2 days post surgery, then begin to gain weight by the third day post surgery. Orally fed rats with 80% jejunoileal resection lose weight for a variable period of time, 1 to 3 days after surgery, then begin to gain weight 2 to 4 days after surgery (Vanderhoof, et al. (1992) Gastroenterol. 102:1949-1956; Lemmey, et al. (1991) Am. J. Physiol. 260:E213-E219) . Rats given liposome-encapsulated oxymorphone before intestinal resection had higher post-surgical body weights compared with animals given standard oxymorphone every 4-8 hours. In Experiment 1 comparing liposome-encapsulated oxymorphone to standard oxymorphone, rats with intestinal resections given liposome-encapsulated oxymorphone had less weight loss from days 0 to 3 (before surgery to day 3 post surgery) compared to rats that were given standard oxymorphone. These rats also started gaining statistically significant amounts of body weight between their first and third day post surgery. Similar results were obtained for the resected rats in Experiment 2. In Experiment 1, the body weight data at 7 days after surgery showed the same data trend as that seen at 3 days. Also, similar data trends were observed for rats with intestinal transections, but did not reach statistical significance because of the smaller numbers in each group of transected rats.

Daily food consumption was also measured in the visceral pain model. In Experiment 1, there was no significant difference in cumulative food consumption for resected rats that had been given 1.2 mg/kg liposome-encapsulated oxymorphone hydrochloride compared to rats that had resections and were treated with 0.3 mg/kg standard oxymorphone every 4 hours. However, in Experiment 2, rats given 1.6 mg/kg liposome-encapsulated oxymorphone had significantly higher food consumption than rats given 0.3 mg/kg standard oxymorphone every 8 hours. A similar data trend was observed for rats that had intestinal transections, but the data did not reach statistical significance because of the lower numbers of animals in each group.

Urine production was measured daily for the first three days post surgery. The general pattern of urine production was similar for all groups. More urine was produced the first day post surgery, but declined sharply over the next 2 days. Rats (resected or transected) that had been given liposome-encapsulated oxymorphone produced less urine on day 1 than rats that had been given standard oxymorphone, but this difference was not statistically significant (p=0.21) . Urine production was similar in these rats on days 2 and 3 post surgery. At no time during these experiments did any of the rats have clinically significant urinary retention necessitating manual expression of the bladder.

In the visceral pain model, there was no significant difference between the concentrations of oxymorphone measured in urine between rats given one subcutaneous injection of liposome-encapsulated oxymorphone or repeated injections (either every 4 or every 8 hours) of standard oxymorphone. Both preparations were detectable in urine up to 72 hours after surgery. Based on results of this study, liposome-encapsulated oxymorphone relieved post-operative visceral pain for at least 48 hours.

Studies of standard pharmaceutical and liposomal preparations injected in dogs indicated no effect on heart rate, respiratory rate, or temperature of the liposome-encapsulated oxymorphone compared to injection of standard oxymorphone preparations. Further, no local skin reactions at the site of injection were observed in any dog.

Sedation scores at both low (0.5 mg/kg liposome-encapsulated oxymorphone; 0.05 mg/kg standard oxymorphone) and high doses (1 mg/kg liposome-encapsulated oxymorphone; 0.1 mg/kg standard oxymorphone) of drug peaked at 30 minutes in dogs that received standard oxymorphone, and at 1 hour in dogs that received the liposome-encapsulated oxymorphone. Sedation scores were not different at any time points except 1 hour after drug administration, when sedation scores for the dogs that received 1.0 mg/kg liposome-encapsulated oxymorphone were significantly higher for an equipotent dose of the standard preparation.

A moderate and statistically insignificant decrease in respiratory rate and rectal temperature were observed in dogs that received either dose of the liposome-encapsulated oxymorphone preparation or the standard preparation of oxymorphone compared to the baseline rate. A significant decrease in heart rate was observed from baseline to 2 hours after drug administration, but actual heart rates were considered to be within a clinically normal and acceptable range.

In dogs that received 1.0 mg/kg of liposome-encapsulated oxymorphone, serum concentrations persisted out to 5 days after drug administration, and were equivalent at 3 days to those seen in dogs that received 0.1 mg/kg of the standard oxymorphone preparation at 4 hours after drug administration. Detectable urine concentrations of oxymorphone persisted out to 7 days, indicating that metabolites of oxymorphone may have had analgesic effects as well.

Table 1 summarizes the pharmacokinetic estimates for the dog model for the doses and formulations administered.

TABLE 1

| | | | Liposome- | | |
|---|---|---|---|---|---|
| | Standard | | Encapsulated | | |
| | Pharmaceutical Dose | | Oxymorphone Dose | | |
| | Group (mg/kg) | | Group (mg/kg) | | |
| | 0.05 | 0.1 | 0.5 | 1.0 | Combined |
| Parameter | (N = 7) | (N = 6) | (N = 6) | (N = 6) | N = 25 |
| K12 (hr⁻¹) | N/A | N/A | N/A | N/A | 0.0165 |
| K23 (hr⁻¹) | 0.400 ± | 0.348 ± | 0.422 ± | 0.356 ± | 0.382 ± |
| | 0.069 | 0.0517 | 0.0406 | 0.081 | 0.066 |
| K30 (hr⁻¹) | 5.40 ± | 7.19 ± | 7.42 ± | 5.67 ± | 6.38 ± |
| | 1.50 | 0.80 | 1.72 | 2.84 | 1.96 |
| Fl (%) | N/A | N/A | 90.9 ± | 87.1 ± | 89.0 ± |
| | | | 5.1 | 1.2 | 4.2 |
| V (L) | 2.03 ± | 1.50 ± | 1.75 ± | 1.09 ± | 1.61 ± |
| | 0.71 | 0.64 | 0.66 | 0.05 | 0.65 |
| AUC | 128.0 | 135.9 | 20.5 | 78.1 | 103.1 |
| (ng-hr/L)/ | (111.6, | (68.5, | (17.1, | (42.2, | (34.8, |
| mg/kg) | 141.8) | 208.7) | 22.4) | 178.7) | 150.1) |

There were no significant differences between any. of the treatment groups. Data from animals that were treated with both preparations are included in the respective group.
F1 represents the dose fraction available in the liposome.
Kᵢⱼ: first order drug transfer rates.
K12: from liposome into subcutaneous tissue.
K23: from subcutaneous tissue to systemic tissue of volume V.
K30: from systemic tissue of volume V to excretion from body.
N/A: K12 was assumed to be a physicochemical constant and was not allowed interpatient variability. By definition, there was no delivery of aqueous drug to the liposomal compartment. AUC (Area Under the Curve) data were non-normal, and are reported as median and 25%, 75% percentiles.

The pharmacokinetic modeling indicates that 90% of the liposome-encapsulated dose administered was in the liposomes, with only 10% of the drug on average having leaked into the aqueous phase prior to administration. Although animal weight proved to be a significant covariate for distribution volume, the inclusion of animal sex as a covariate did not improve the model.

ANOVA analysis of the effect of dose and formulation upon the plasma oxymorphone AUC for the two doses for each oxymorphone preparation indicated that the dose-adjusted AUC for the two standard and 1 mg/kg liposome-encapsulated doses are not significantly different. However, the 0.5 mg/kg dose of liposome-encapsulated oxymorphone had a significantly lower dose-adjusted AUC than the other groups. The t_{1/2} of liposome-encapsulated oxymorphone was 42 hours.

The pharmacokinetics of the subcutaneously administered standard and liposomal oxymorphone are linear for the higher dose of liposome-encapsulated drug and a disproportionately low amount of drug is available from the lower (0.5 mg/kg) dose. It is possible that the sensitivity of the ELISA assay used was not high enough to accurately measure the much lower concentrations of oxymorphone provided to the plasma from the lower dose of liposome-encapsulated drug. Concentrations at or below 1 ng/ml were discarded as being within the mean plus one standard deviation of the concentrations measured in plasma taken from animals prior to treatment. It is likely that a more sensitive assay would improve the ability to characterize the AUC arising from the lower doses. Further, a finite amount of drug may be trapped within the liposome and the absorption of this portion of drug into blood significantly may be delayed. The amount of drug sequestered in such a manner would constitute a greater fraction of a lower dose than a larger one.

The variability of the fraction of oxymorphone in the liposomal preparation that was delivered in the aqueous phase was quite consistent (89.6%±1.82%(SE)). This consistency indicates that this fraction was not markedly affected by differences in storage time or time at refrigerator or room temperature prior to administration. The rate of flux of oxymorphone out of the liposome was 0.165/hour. Based on t½ and rate of flux of drug out of the liposome, this liposome-encapsulated oxymorphone preparation could be dosed once to twice weekly in dogs. Dosing recommendations for standard (non-liposome-encapsulated) oxymorphone in dogs are every 4 hours. Therefore, liposome-encapsulation of oxymorphone greatly extends the duration of drug effect and makes this formulation potentially useful for treatment of chronic pain in dogs.

Parrots receiving a subcutaneous injection of 10 mg/kg liposome-encapsulated butorphanol had sustained levels of butorphanol in the blood for 24 hours after injection and had detectable levels of butorphanol at eight days after injection (Table 2).

TABLE 2

| | Amount of Butorphanol |
|---|---|
| Time | (ng/ml) |
| 5 minutes | 74.8 |
| 15 minutes | 75.3 |
| 30 minutes | 74.8 |
| 1 hour | 75.2 |
| 2 hours | 75.2 |
| 12 hours | 71.4 |
| 24 hours | 70.5 |
| 48 hours | 31.4 |
| 72 hours | 29.9 |
| 120 hours | 9.8 |
| 192 hours | 14.2 |

Amount of butorphanol represents the average of three birds per time point minus the baseline amount.

The present invention is therefore a method for providing for long-term analgesic activity, without multiple drug administrations, by administering to an animal, including humans, an effective dose of a liposome-encapsulated opioid formulation, including but not limited to oxymorphone, butorphanol, morphine and hydromorphone, so that analgesia is produced for a longer period of time than analgesia that results from administration of an effective dose of a non-liposome-encapsulated opioid formulation. In general, long-term analgesic activity is meant as a period of time greater than or equal to 24 hours after administration of a single dose of a liposome-encapsulated opioid formulation provided herein. In the context of the present invention, “an effective dose” is a dose of the analgesic drug known to have activity to decrease pain in animals, including humans. Pain may be measured by assessing behavioral hypersensitivity. Behavioral hypersensitivity of pain may include sensations that are sharp, aching, throbbing, gnawing, deep, squeezing, or colicky in nature and may be measured by, for example, exposure to thermal hyperalgesia or mechanical hyperalgesia. One of skill would choose such an effective dose based on the results of in vivo studies of the drug when administered alone or on data showing pharmacological activity in cells or animals, including humans. It is contemplated that the liposome-encapsulated opioid may be administered at delayed intervals, i.e., every few days to once a week. The present invention is therefore also a method for reducing dose-limiting toxicity of an opioid analgesic drug, including but not limited to oxymorphone, which comprises administration of the drug in the liposome formulation of the present invention wherein administration of the liposome-encapsulated drug results in a reduction in observed adverse effects in animals treated with liposome-encapsulated drug as compared to animals treated with non-liposome-encapsulated drug. Other opioid compounds may be formulated with this method as shown by the fact that morphine, butorphanol, and hydromorphone have each been effectively formulated using the method of the present invention. One of skill would choose the opioid to be formulated from those approved for use in either animals or humans.

The invention is described in greater detail by the following non-limiting examples.

### EXAMPLE 1

### Preparation of Liposome-Encapsulated Oxymorphone using Dehydration/Rehydration Vesicles

For the preparation of liposome-encapsulated oxymorphone, 42.4 mg powdered oxymorphone free base (Oxymorphone free base, USP, Rockville, Md.) was dissolved in 10 ml of 1 mM sodium citrate buffer pH 4.0 to form a 12 mM solution of oxymorphone HCl. The solution was sterilized using a 0.22 μm filter (Gelman Filters, Pall Corp., Ann Arbor, Mich.). A film containing 80 μmol of egg phosphatidylcholine was dried onto the walls of an 18×160 mm screw-capped culture tube using a rotary evaporator, with water bath at 37° C. The dried film of egg phosphatidylcholine was suspended in 5 ml of the oxymorphone hydrochloride solution. The mixture was sonicated in a cylindrical sonic bath (Laboratory Supplies Co., Hicksville, N.Y.) for a total of 10 minutes. The resulting mixture was transferred to sterile round-bottomed flasks, frozen using a dry ice and isopropanol bath, and lyophilized for 24-48 hours. The lyophilized preparation was stored at −20° C. until use.

### EXAMPLE 2

### Preparation of Liposome-Encapsulated Morphine using Multivesicular Liposomes

Lipid mixtures were prepared according to established methods (Kim, et al. (1983) Biochim. Biophys. Acta 728:339-348). The lipid mixture contained: 1,2-dipalmitoyl-sn-glycero-3-[phospho-rac-(1-glycerol)] (sodium salt) (Avanti Polar Lipids, Alabaster, Ala.), cholesterol (Avanti Polar Lipids, Alabaster, Ala.), triolein (Sigma Chemical, St. Louis, Mo.), 1,2-dioleoyl-sn-glycero-3-phosphocholine (Avanti Polar Lipids, Alabaster, Ala.), chloroform (Spectrum Chemical, Gardena, Calif. and New Brunswick, N.J.), and distilled sterile water for irrigation.

### EXAMPLE 3

### Assay for In Vitro Release Kinetics of Liposome-Encapsulated Oxymorphone

Cellulose dialysis tubing was prepared by boiling in EDTA and stored in sterile physiologic saline (Yu, et al. (1999) J. Invest. Dermatol. 112:370-375). Sections of tubing were filled with either 0.319 μl of normal rat serum and 0.181 μl of oxymorphone hydrochloride liposomes suspended in saline-acetate buffer or 0.181 μl of saline-acetate buffer alone. The end of the dialysis tubing was tied off and the bag was placed in 9.5 ml of saline for irrigation in a 50 ml conical centrifuge tube. Tubes were prepared in duplicate and then placed on a rotary shaker at 20° C. At 3 hours and then daily for 7 days, an aliquot of buffer was removed, placed in a cuvette and the absorbance determined at 281 nm. The dialysis bag was then transferred to a fresh tube containing 9.5 ml of saline for irrigation and placed on the shaker (Woolf and Chong (1993) supra).

### EXAMPLE 4

### In Vivo Pharmacokinetics in the Neuropathic Pain Model

Experiments were performed on male Sprague-Dawley (Harlan, Madison, Wis.) rats weighing between 275-300 g. All rats were tested to be free of respiratory andenteric bacteria, ecto- and endo-parasites, and the following serological antigens: Mycoplasma pulmonis, Sendai, SDAV/RCV, KRV, H-1, RPV, PVM, Reo-3, Hantaan, TMEV, MAd, LCM, Clostridium piliforme, and E. cunicului. The rats were housed in polypropylene caging and provided commercial rat diet (Harlan Teklad Laboratory Rodent Diet, pelleted) and water ad libitum. All surgical procedures were performed under isoflurane anesthesia administered via face mask to effect. A well-established chronic constriction injury model (Bennett and Xie (1988) Pain 33:87-107) was used to induce thermal hyperalgesia in the test animals. Briefly, the left sciatic nerve was exposed and freed from underlying connective tissue, and four loose ligatures of 4-0 chromic gut were placed around the nerve. Ligatures did not cause the suture material to visibly tighten around the nerve sheath. The onset of hyperalgesia in this model is thought to be due to inflammatory changes that occur in the sciatic nerve within hours of placing the ligatures (Bennett and Xie (1988) supra) . Overlying muscle was closed with simple continuous sutures of 4-0 chromic gut, and the skin was closed with surgical skin staples. Rats with sciatic ligation exhibit near-maximal thermal hyperalgesia by 3-7 days following surgery (Smith, et al. (2002) Pain 97:267-273). Beyond 7 days, thermal hyperalgesia begins to decrease. In control (non-ligated) animals, anesthesia was induced and liposome-encapsulated drug or sucrose liposomes were injected as in the ligated rats but no surgery was performed. All experiments and surgical procedures were conducted in accordance with guidelines accepted by the International Association for the Study of Pain (Zimmerman (1983) Pain 16:109-110). Any animal that exhibited signs of severe pain (autotomy, anorexia, lack of grooming or social interaction) was to be removed from the study immediately and euthanized with an overdose of pentobarbital IP. In the current study, humane euthanasia was not necessary in any of the rats.

Baseline hindpaw thermal withdrawal latencies were established for all animals before they were randomly and equally divided into six groups. The rats were habituated to the testing device (Ugo-Basile, Milan, Italy) for 15 minutes prior to testing. Thermal sensitivity was measured using paw withdrawal latency to a radiant heat stimulus (Hargreaves, et al. (1988) Pain 32:77-88). Paw withdrawal latency was determined as the average of four to five measurements over a 30-minute test period. Stimulus intensity and rate of heating of the thermistor was kept constant throughout the entire experiment, and was adjusted to give approximately a 9-10 second withdrawal latency in a normal, non-injured animal. Only the left (affected) hind paw was tested.

After baseline thermal withdrawal latencies were determined, test animals (sciatic nerve ligation performed) were divided into three groups: Group 1 (n=6) liposome-encapsulated sucrose (0.8 ml); Group 2 (n=6) liposome-encapsulated oxymorphone (1.2 mg/kg); Group 3 (n=6) liposome-encapsulated morphine (2.8 mg/kg) . After baseline thermal withdrawal latencies were determined control animals (non-ligated) were divided into three groups: Group 4 (n=8) liposome-encapsulated sucrose (0.8 ml), Group 5 (n=8) liposome encapsulated morphine (2.8 mg/kg) and Group 6 (n=8) liposome-encapsulated oxymorphone (1.2 mg/kg). A minimum number of six animals per group was necessary to achieve adequate statistical power (Backonja, et al. (1995) Neurosci. Lett. 196:61-64; Smith, et al. (2002) supra) . A minimum number of animals was used in an attempt to subject the fewest number of animals necessary to a chronically painful condition, i.e. hyperalgesia. While under isoflurane anesthesia, but before sciatic nerve ligation in the test animals, rats were administered a single subcutaneous injection of either liposome-encapsulated sucrose, morphine, or oxymorphone depending on their group assignment. The skin over the right quadriceps muscle was shaved and all subcutaneous injections were given at that site in order to assure consistency of drug absorption and to assess any potential skin reactions that might occur. The dose of morphine and oxymorphone chosen was based on previously reported dosage recommendations for rodents (Hawk and Leary (1999) supra) . In general, doses for liposome-encapsulated preparations of drugs are 10 times that of standard formulations.

In two separate age- and sex-matched groups of rats, serum and urine were collected for 7 days to assess the duration of detectable oxymorphone concentrations after one injection of liposome-encapsulated oxymorphone. Male Sprague-Dawley (Harlan, Madison, Wis.) rats weighing −300 g. were administered one subcutaneous injection of liposome-encapsulated oxymorphone synthesized as described above at 1.2 mg/kg (n=6). Rats were housed in metabolism cages to facilitate urine collection. Blood was collected from the ventral tail artery or jugular vein under isoflurane anesthesia administered by face mask. Blood was collected at baseline, 4 hours, and 1, 2, 3, 4, and 7 days after drug administration. Urine was collected at 1, 2, 3, 4, 5, 6, and 7 days after drug administration. In a separate age and weight matched control group (n=3) urine was collected from untreated rats housed in metabolism cages to determine the contribution of background interference due to the method of urine collection on the oxymorphone assay. Serum and urine samples were frozen at −70° C. until analysis. Oxymorphone concentrations were assayed using a commercial ELISA kit specific for oxymorphone detection (Neogen Inc., Lexington, Ky.).

Repeated measures ANOVA was used for data analysis. The main emphasis was on detecting differences in thermal withdrawal latencies both within and between groups. All significant effects were further analyzed with Scheffe's test. Significance was inferred at the p<0.05 level.

### EXAMPLE 5

### Small Intestinal Resection Model of Short Gut Syndrome

Male Sprague Dawley rats (Harlan Sprague Dawley, Madison, Wis.) weighing 250 grams were used to test the analgesic efficacy of liposome-encapsulated oxymorphone in a model of short gut syndrome. The rats used in this experiment were housed in wire-bottomed cages to facilitate food consumption measurements, including spillage, and urine collection. Rats were fed a powdered high tat diet (Vanderhoof, et al. (1992) supra; Lemmey, et al. (1991) supra) and water ad libitum. Rats were administered liposome-encapsulated oxymorphone and saline injections or blank sucrose liposomes and injections of standard pharmaceutical oxymorphone (NUMORPHAN®, Endo Pharmaceuticals, Chadds Ford, Pa.).

Intestinal surgery was performed using well-known methods (Clark, et al. (2001) Contemp. Topics Lab. Anim. Sci. 40:21-26.35). Rats were anesthetized with isoflurane in oxygen delivered by mask and given dosages of analgesic drugs as described below. Rats were prepared for aseptic abdominal surgery. The internal viscera were exposed through a ventral midline incision. Rats given intestinal resections underwent a 70% mid jejunoileal resection commonly used to study intestinal adaptation in orally-fed rats (Vanderhoof, et al. (1992) supra; Lemmey, et al. (1991) supra; Clark, et al. (2001) supra) . Resected animals had bowel removed from 15 cm distal to the ligament of Treitz until 15 cm proximal to the cecum. The jejunum and ileum were measured using a 15 cm length of sterile silk suture placed along the anti-mesenteric border of the bowel so that all resected animals had an equivalent amount of proximal jejunum (15 cm) and distal ileum (15 cm) remaining. In transected animals, the ileum was severed 15 cm proximal to the cecum. Intestinal transections and resections were closed as an end-to-end anastomosis with 6-0 silk suture. The abdominal incision was closed using continuous sutures in the body wall and skin staples.

Rats were randomly assigned to two groups in Experiment 1 and two groups in Experiment 2. In Experiment 1, Group L1 rats (n=8) received subcutaneous injections of 1.2 mg/kg liposome-encapsulated oxymorphone and a subcutaneous injection of saline prior to surgery. Additional subcutaneous dosages of 0.1 ml of saline were given to Group L1 rats every 4 hours for 24 hours after surgery. Group S1 rats (n=8) received subcutaneous injections of 0.15 mg/kg of the standard pharmaceutical preparation of oxymorphone and a subcutaneous injection of 0.1 ml of sucrose liposomes prior to surgery. Additional subcutaneous dosages of 0.3 mg/kg standard oxymorphone were given to Group S1 rats every 4 hours for 24 hours after surgery. In Experiment 2, Group L2 rats (n=8) received subcutaneous injections of 1.6 mg/kg liposome-encapsulated oxymorphone and a subcutaneous injection of 0.1 ml of saline prior to surgery. Additional subcutaneous dosages of 0.1 ml of saline solution were given to Group L2 rats every 8 hours for 24 hours after surgery. Group S2 rats (n=6) received a subcutaneous injection of 0.3 mg/kg of standard oxymorphone and a subcutaneous injection of 0.1 ml of sucrose liposomes. Additional dosages of 0.3 mg/kg standard oxymorphone were given to Group S2 rats subcutaneously every 8 hours for 24 hours after surgery. In all groups in both experiments, subcutaneous injections of oxymorphone or sucrose liposomes were given in the flank region. Subcutaneous injections of standard oxymorphone or saline solution were given between the shoulder blades. Rats were also given prophylactic antibiotics. Fifty mg of ampicillin was administered subcutaneously prior to surgery. Additional dosages were given every 12 hours for 48 hours after surgery. Experiment 1 rats were euthanized 7 days after surgery. Experiment 2 rats were euthanized 3 days after surgery.

Postoperative pain in rats was assessed using a modified behavioral scoring system (behavioral ethogram) (Clark, et al. (2001) supra) . Observations occurred every 4 hours for the first 24 hours after surgery and every 8 hours up to 48 hours after surgery. Observers recording numerical pain scores also gave doses of saline or oxymorphone to each rat immediately after behavioral observations were complete. Observers were blinded to the treatment condition of each rat and did not know if they were administering injections of saline or oxymorphone.

In addition to ethographic measurements of pain, several other measurements were included to assess well-being in rats after intestinal resection or transection. Rats were weighed and food consumption was measured daily. Also, urine was collected and the volume measured every 24 hours for 72 hours after surgery.

Urine was obtained by housing rats in metabolism cages. Twenty-four hour urine collections were obtained for each rat on days 1 to 3 after surgery. Urine samples were frozen at −70° C. until analysis. Oxymorphone concentrations were assayed using a commercial ELISA kit specific for oxymorphone detection (Neogen Inc., Lexington, Ky.).

Behavioral scoring data was analyzed using a 2-way ANOVA design with replication using the Statmost software program (Statmost, Dataxiom Software Inc., Los Angeles, Calif.). Other data, including urine volumes, body weight, and food consumption were analyzed using a Student's t test on the Excel software package (Excel, Microsoft Inc., Redmond, Wash.).

### EXAMPLE 6

### In Vivo Pharmacokinetics in Dogs

Twenty-five adult beagles (n=14; 12 female, 2 male) and hounds (n=11; 9 female, 2 male) that were 1 to 5 years old (median age 1.5 years) and weighed between 6.9 kg and 35.7 kg (median weight=9.46 kg) were used. Normal health status and organ function were confirmed by physical exam and a complete blood count and serum chemistry profile prior to inclusion in the study. None of the animals received any medications in the 2 weeks prior to study. Animals were housed individually (hounds) or in groups of 2 to 4 (beagles), and were fed a commercial diet and water ad libitum throughout the study.

Two doses of liposome-encapsulated oxymorphone and two equipotent doses of standard pharmaceutical oxymorphone were investigated. One dose of the liposomal vehicle was also investigated to confirm the absence of pharmacodynamic properties of the vehicle. Each dog received only 1 treatment and was then removed from further study. Treatment groups were as follows: Group 1 (n=6) liposome-encapsulated oxymorphone (1.0 mg/kg); Group 2 (n=5) liposome-encapsulated oxymorphone (0.5 mg/kg); Group 3 (n=6) standard pharmaceutical oxymorphone (NUMORPHAN®, Endo Pharmaceuticals Inc., Chadds Ford, Pa.) (0.1 mg/kg); Group 4 (n=6) standard pharmaceutical oxymorphone (0.05 mg/kg); Group 5 (n=2) liposomal vehicle (0.7 ml). Liposome-encapsulated oxymorphone or vehicle were drawn from a stock solution on ice via an 18 gauge needle attached to a 1- or 3-ml syringe and administered subcutaneously slowly via a 23 gauge needle. All injections were made in a shaved area of the thigh such that potential skin reactions to the liposomal preparations could be assessed.

A 1 ml blood sample was collected percutaneously from the left and right cephalic or saphenous veins, in rotating order. In groups 1 and 2 (liposome-encapsulated oxymorphone groups), blood was collected before and 0.5, 1, 2, 4, 8, 12, 16, and 24 hours after drug administration, and daily for 5 days at the same time of day as treatment administration had occurred. In groups 3, 4, and 5 (standard oxymorphone and liposomal vehicle groups), blood was collected before and 0.5, 1, 2, 4, 8, 12, and 24 hours after drug administration. Blood samples were transferred to centrifuge tubes and held on ice for a maximum of 4 hours, then centrifuged at 5° C. for 10 minutes at 5000×g. Serum was stored at −70° C. until analysis.

A sedation score was recorded on each dog prior to drug administration and at the same times as venous blood collection. The sedation score included recording of heart rate by auscultation, respiratory rate by observation of movement of the thoracic wall, and rectal temperature via a digital rectal thermometer. The same person assessed sedation scores throughout the study. Changes in sedation score, heart rate, respiratory rate, and temperature were analyzed using repeated measures ANOVA, with p<0.05 considered significant.

Oxymorphone concentrations in serum were quantitated using a commercial ELISA (Neogen Inc, Lexington, Ky.).

Plasma oxymorphone concentrations were used in pharmacokinetic model building using a non-linear, mixed effect computer program (NONMEM User's Guides, (1989-98) Beal and Sheiner (Eds.) GloboMax, LLC, Hanover, Md.). The data from all animals were evaluated concurrently. The assumption was made that the subcutaneous injection of the standard and liposomal oxymorphone delivered drug to an extravascular compartment from which unidirectional absorption into the blood occurred. In the case of the liposomal preparation, an additional, unidirectional rate constant of flux from the liposome to the surrounding subcutaneous compartment was assumed.

The rapid appearance of oxymorphone in plasma at the first sample drawn 30 minutes after injection of the liposomal preparation and occasional double concentration peaks indicated that some of the liposomal-encapsulated oxymorphone may have leaked into the aqueous diluent. This was tested in the pharmacokinetic model construction with allowance for a variable (fitted) fraction of the liposomal dose administered directly to the subcutaneous space in the aqueous carrier.

The NONMEM model included an intersubject variability for the parameters of distribution volume, fraction of drug delivered to of the liposome, and drug flux into and out of the plasma. The first-order rate of drug loss from the liposome was assumed to be a physiochemical constant, and was not allowed to vary between animals. Animal weight and sex were tested as continuous and categorical covariates affecting volume and clearance. Pharmacokinetic models that included a tissue distribution compartment were over-parameterized and were discarded.

In addition to the NONMEM modeling, the AUC (Area Under the Curve) oxymorphone was calculated using the trapezoidal method over the 24 or 120 hours following the standard or liposomal preparations, respectively. Concentrations less than 1 ng/ml were considered to be not different than zero in the computations. This threshold was taken as the mean +1 standard deviation of the oxymorphone concentrations measured in the pre-dose plasma samples from these opioid-naive animals.

The AUC from last measured plasma concentration to infinity was extrapolated using the actual concentration and the animal-specific elimination rate constant derived from the NONMEM analysis. Comparison of the AUCs was done after normalizing the values to the dose of oxymorphone administered.

What is claimed is:

1. A liposome-encapsulated opioid formulation comprising an opioid compound encapsulated in a liposome by a rehydration/dehydration method that produces liposome-encapsulated opioid so that said liposome-encapsulated opioid has long-term analgesic activity in an animal.

2. The liposome-encapsulated opioid formulation of claim 1, wherein the opioid compound comprises oxymorphone, morphine, butorphanol, or hydromorphone.

3. The liposome-encapsulated oxymorphone formulation of claim 2, wherein the liposome-encapsulated oxymorphone is produced with an efficiency of at least 30°.

4. A method for producing liposome-encapsulated opioid comprising:
a) suspending opioid in a buffer to form an oxymorphone-buffer mixture;
b) overlaying the opioid-buffer mixture onto a film of lipid;
c) sonicating the opioid-buffer mixture and the lipid to form liposome;
d) freezing the liposomes by mixing the liposomes over a slurry of dry ice and isopropanol; and
e) freeze-drying the liposomes for storage in a freezer until the liposome mixture is rehydrated in sterile water.

5. A liposome-encapsulated opioid produced in accordance with the method of claim 4.

6. A method for producing liposome-encapsulated opioid comprising:
a) suspending opioid in a buffer to form an oxymorphone-buffer mixture;
b) overlaying the opioid-buffer mixture onto a film of lipid;
c) freezing the opioid-buffer mixture and film of lipid; and
d) thawing the mixture to generate liposomes.

7. A liposome-encapsulated opioid produced in accordance with the method of claim 6.

8. A method of producing long-term analgesic activity in an animal comprising administering to an animal an effective dose of the liposome-encapsulated formulation of claim 1 so that analgesia is produced for a longer period of time than analgesia that results from administration of an effective dose of a non-liposome-encapsulated formulation.

9. A method for reducing the dose-limiting toxicity of oxymorphone hydrochloride in an animal comprising administering to an animal an effective dose of the liposome-encapsulated oxymorphone formulation of claim 2.
